(19) United States
(12) Patent Application Publication
(10) Pub. No.: US 2012/0148501 A1
(43) Pub. Date: Jun. 14, 2012

(54) RADIOLOGICAL IMAGE ENHANCEMENT WITH TANTALUM CLUSTERS

(76) Inventors: Fred Geisler, Aurora, IL (US); Marc Schrier, El Granada, CA (US); Mihai Buretea, San Francisco, CA (US)

(21) Appl. No.: 12/962,965

(22) Filed: Dec. 8, 2010

(51) Int. Cl. A61K 49/04

(52) U.S. Cl. 424/9.42

(57) ABSTRACT

A solution comprising a defined concentration of purified tantalum clusters in a solvent selected from the group consisting of water, ethanol, ethylene glycol and propylene glycol; wherein said defined concentration is greater than 100 mM, preferably greater than 150 mM; most preferably greater than 300 mM. The purified tantalum clusters are obtained by sequentially washing crude tantalum clusters containing residual chloride ions with aqueous hydrochloric acid to remove residual sodium chloride; and washing the hydrochloric acid-washed tantalum clusters with diethyl ether to remove residual hydrochloric acid and water.

### BACKGROUND

This disclosure relates generally to enhancement of X-ray images using heavy metal-based reagents. More particularly, this disclosure relates to enhancement of X-ray images using tantalum-based inorganic clusters.

Approximately 75% of the 300 million diagnostic examinations performed annually in the United States are X-ray based. Contrast agents are often used to allow visual imaging of vessels and tissues. These contrast agents contain one or more high-Z (high atomic number) atoms. The greater the contrast, the better the ability of the radiologist to identify a region of interest (ROI) against a background of normal surrounding tissues, while reducing the overall patient radiation dose. The current state of the art contrast agents for vascular imaging are based on iodinated (Z=53) compounds. A new X-ray contrast agent based on a higher Z element such as tantalum (Z=73) would have the advantages of higher intrinsic contrast coupled with lower radiation exposure to patients. It would also solve current problems, such as iodinated agents' inadequate contrast for some procedures, especially in examinations of obese patients, and provide a contrast agent for patients allergic to iodine. A tantalum based contrast agent would also allow a physics-optimal higher incident X-ray energy which would even further enhance the visualization of vascular structures by decreasing the density of the bony images in the ROI.

Agents containing one or more high-Z atoms offer substantial improvements in contrast and spatial resolution, while reducing the overall patient radiation dose. Tantalum (Ta) has been used for decades as both a metallic implant and radiographic marker. The ideal tantalum-based contrast agent (1) provides high X-ray attenuation and therefore high contrast, (2) is stable under physiological conditions, (3) is available in high concentration, (4) is nontoxic, (5) is injected easily, (6) resides in the ROI (Region of Interest) for a sufficient length of time to allow imaging, and (7) is then completely excreted from the body. Cluster compounds that contain several high-Z atoms, preferably several tantalum atoms, enable high X-ray attenuation, and are therefore of particular interest.

A tantalum based contrast agent may offer visual contrast which is as good as, or superior to, current iodine based agents in most routine radiological applications using current radiologic equipment. Since equivalent visualized contrast is produced with a smaller total quantity of a tantalum agent, the decreased total volume injected may yield improved patient safety and comfort. Finally, as tantalum has a greater X-ray attenuation at higher X-ray photon energies compared to iodinated agents, the overall radiation exposure can be reduced for equivalent visual images on the radiographics.

Multinuclear clusters for use as metal complex X-ray contrast agents have been identified in the prior art. Although there has been speculation that tantalum-based clusters may be used as contrast agents, most of the known examples describe complexes of metals such as tungsten. Many of these compounds are soluble in organic solvents, but are insoluble or poorly soluble in water. Insoluble clusters must therefore be made into aqueous emulsions through the use of an organic emulsifier.

It has been demonstrated that intra-arterial injection of a 500 mM solution of a soluble tungsten cluster into a rat paw and forearm produced excellent visualization of the circulatory system. Blood vessels which were not seen upon injection of an equal volume of a higher concentration (920 mM) of an iodine-based contrast agent were clearly visualized with the tungsten cluster.

Although the potential for use of tantalum clusters having Ta₆Cl₁₂ cations as X-ray contrast agents has been discussed, the current state of the art suggests that these compounds are insufficiently water-soluble to be effective contrast agents. The prior art fails to show that an inorganic multinuclear metal complex of tantalum may be made water soluble and used as a contrast agent.

It is an object of various embodiments disclosed herein to provide an improved low-dose X-ray contrast agent based on tantalum clusters.

It is a further object of various embodiments disclosed herein to provide an improved low-dose X-ray contrast agent based on water-soluble tantalum clusters.

It is an additional object of various embodiments disclosed herein to provide an improved low-dose X-ray contrast agent based on inorganic tantalum clusters.

The foregoing objects and advantages are illustrative of those that can be achieved by the various embodiments disclosed herein and are not intended to be exhaustive or limiting of the possible advantages that can be realized. Thus, these and other objects and advantages of the various exemplary embodiments will be apparent from the description herein or can be learned from practicing the various exemplary embodiments, both as embodied herein or as modified in view of any variation that may be apparent to those skilled in the art. Accordingly, the present invention resides in the novel methods, arrangements, combinations, and improvements herein shown and described in various exemplary embodiments.

### SUMMARY OF THE INVENTION

In light of the present need for improved X-ray contrast agents, a brief summary of various embodiments is presented. Some simplifications and omissions may be made in the following summary, which is intended to highlight and introduce some aspects of the various embodiments, but not to limit the scope of the invention. Detailed descriptions of preferred embodiments adequate to allow those of ordinary skill in the art to make and use the subject matter disclosed herein will follow in later sections.

Various embodiments described in the current disclosure relate to a solution comprising a defined concentration of tantalum clusters having the formula:

(Ta₆₋ₓMₓX₁₂)L_{y}A_{z},

where M is selected from the group consisting of zinc, niobium, and tungsten where x can have a value in the range of 0 to 6. X is selected from the group consisting of chlorine, bromine, iodine, oxygen, sulfur, selenium, and tellurium. L is an inorganic or organic ligand, which may be a charged ligand or an uncharged ligand. Typical uncharged ligands include, for example, water, methanol or ethanol. In these tantalum clusters, A is an inorganic counterion like Cl⁻¹, Bf⁻¹, I⁻¹, SO₄⁻², NO₃⁻¹, PO₄⁻³, or OH⁻¹. The solution comprises a solvent selected from the group consisting of water, an aqueous solution of Polysorbate 80, ethanol, ethylene glycol, propylene glycol and the defined concentration of tantalum clusters is greater than 120 mM.

The tantalum clusters having the formula (Ta₆₋ₓMₓX₁₂)L_{y}A_{z} may be further characterized in that A is Cl, Br, or I. The tantalum clusters may be further characterized in that each X is a halogen atom, preferably in that each X is the same halogen atom. According to various examples disclosed herein, the tantalum clusters may be characterized in that each X is Cl. According to certain embodiments described herein, the tantalum clusters may be synthesized in the presence of dopants, preferably oxygen, sulfur, selenium, or tellurium dopants, giving rise to tantalum clusters having the formula (Ta₆₋ₓMₓX₁₂)L_{y}A_{z} where some of the twelve atoms X are halogen atoms, with the remaining X atoms being oxygen, sulfur, selenium, or tellurium atoms.

Various examples disclosed herein relate to a solution of tantalum clusters having the formula (Ta₆₋ₓMₓX₁₂)L_{y}A_{z}, where A is Cl, Br, or I; and z is between 2 and 3. Where x and z is +2, the cationic cluster has a net charge of +2 and each tantalum atom has a formal charge of +2.33. Where x is zero and z is +3, the cluster has a net charge of +3 and each tantalum atom has a formal charge of +2.5.

In various embodiments of the solution of tantalum clusters, the solvent is water or an aqueous solution of Polysorbate 80, such as a 10% aqueous solution of Polysorbate 80. The concentration of tantalum clusters in such a water-based solution is greater than 120 mM, preferably greater than 150 mM, more preferably greater than 300 mM.

In various embodiments of the solution of tantalum clusters, the solvent is ethylene glycol. The concentration of tantalum clusters in such an ethylene glycol solution is greater than 150 mM, preferably greater than 300 mM, more preferably greater than 500 mM. In alternative embodiment, the solvent is ethanol, and the concentration of tantalum clusters is greater than 120 mM.

Various aspects of the subject matter disclosed herein relate to a process for purifying tantalum clusters having the formula (Ta₆₋ₓMₓX₁₂)A_{z}, where M is selected from the group consisting of zinc, niobium, and tungsten, and x is between 0 and 6. X is selected from the group consisting of chlorine, bromine, iodine, oxygen, sulfur, selenium, and tellurium, with the proviso that X may comprise one or more atoms from the list. A is an inorganic counterion or mixture of anions. The process comprises a step of preparing crude tantalum clusters having a solubility in water of less than 100 mM. In various embodiments, A is Cl, Br, or I; and the crude clusters are contaminated with starting materials, hydrolyzed starting materials, and salts of halide compounds. In certain embodiments, A and X are each Cl; and the crude clusters are contaminated with sodium chloride.

The crude tantalum clusters are purified by dissolving the crude tantalum clusters in water and adding concentrated aqueous hydrochloric acid to precipitate the clusters, separating them from residual sodium chloride which remains partially or completely in solution. This process is repeated until the concentration of sodium chloride has been reduced to desired levels. The resulting precipitated tantalum clusters are then washed with diethyl ether to remove residual hydrochloric acid and water, recovering purified tantalum clusters. The step of washing with diethyl ether and recovering is repeated a plurality of times until the purified tantalum clusters have a solubility in water of greater than 120 mM, preferably greater than 150 mM, more preferably greater than 300 mM.

Other solvents that offer substantial solubility for hydrochloric acid, but not for the tantalum cluster can be substituted for diethyl ether including, but not limited to, dibutyl ether, tetrahydrofuran, and dimethoxyethane. Other mineral acids can be substituted for hydrochloric acid, including, but not limited to, hydrobromic acid, hydrogen iodide, or hydrofluoric acid.

In various embodiments, the crude tantalum clusters may be purified by dissolving the crude tantalum clusters in water and adding concentrated aqueous hydrochloric acid to precipitate the clusters, separating them from residual sodium chloride which remains partially or completely in solution. The resulting precipitated tantalum clusters are subjected to vacuum to remove water and residual hydrochloric acid until the purified tantalum clusters have a solubility in water of greater than 120 mM, preferably greater than 150 mM, more preferably greater than 300 mM.

In various aspects described herein, the crude tantalum clusters are obtained by stirring a mixture of tantalum clusters; sodium chloride, bromide, or iodide; tantalum or aluminum metal; and tantalum oxides, sulfides, selenides, tellurides, chlorides, bromides, or iodides in water to form a solution; filtering the solution to remove insoluble solids; precipitating the tantalum clusters with concentrated hydrochloric acid; and recovering the precipitated solids.

Various embodiments relate to an X-ray contrast agent, comprising a saturated solution of multinuclear tantalum clusters having bridging ligands, where the saturated solution has a concentration of greater than 70 mM, preferably greater than 120 mM, more preferably greater than 150 mM, and most preferably greater than 300 mM. The bridging ligands may be halide ions. Various embodiments relate to a method of obtaining an enhanced X-ray image of a living subject by administering an X-ray contrast agent comprising a saturated solution of multinuclear tantalum clusters having bridging ligands to the living subject. Various embodiments comprise administering the X-ray contrast agent to the living subject by intra-arterial injection.

### BRIEF DESCRIPTION OF THE DRAWINGS

In order to better understand various exemplary embodiments, reference is made to the accompanying drawings, wherein:

FIG. 1 shows the optical absorbance spectra of the (Ta₆Cl₁₂)Cl₂ and the (Ta₆Cl₁₂)Cl₃ clusters;

FIG. 2 shows the X-ray image of aqueous tantalum cluster samples with calibration standards;

FIG. 3 shows the X-ray image of tantalum cluster in ethylene glycol with calibration standards; and

FIG. 4 shows the a comparison of the absorbency of a tantalum cluster solution in ethylene glycol to the absorbencies of water and Visipaque.

### DETAILED DESCRIPTION OF EXEMPLARY EMBODIMENTS

Hexanuclear tantalum cluster include bridging atoms, ions, and ligands. These clusters fall under a class of (Ta₆₋ₓMₓX₁₂)L_{y}A_{z}, where M may be a doping metal such as zinc, niobium, or tungsten. X represents tantalum-tantalum bridging atoms, L represents ligands, and A represents terminal, charge-balancing anions. The twelve tantalum-tantalum edge-bridging atoms, X, tend to be halogens, especially chlorine, but can also be the chalcogens, including oxygen, sulfur, selenium, or tellurium, preferably oxygen or sulfur. Iodine would be ideal as a bridging atom X from a Z-perspective. However, many people are iodine-allergic, so high-Z metal clusters without iodine are of interest for treating individuals who cannot tolerate iodine. Bromine and chlorine are therefore candidates for use as bridging atoms in clusters. Due to the high Z-value of tantalum, the total quantity of the contrast agent necessary to achieve the same contrast effect as a non-metallated iodinated agent can be reduced. The tantalum atoms in the hexanuclear tantalum clusters range in formal oxidation states from 2.33 to 2.5.

The tantalum atoms in the hexanuclear tantalum clusters range in formal oxidation states from +2 to +3. In various exemplary embodiments, the tantalum clusters contain 6 tantalum atoms, each having an average formal oxidation state of between +2 to +3, preferably between 2.33 and 2.66, more preferably between 2.33 and 2.5. The resulting clusters of formula (Ta₆₋ₓMₓ1ᵢ₂), where x is 0 and X is a halide anion, have a charge of between +2 (average formal oxidation state of tantalum atoms=+2.33) and +4 (average formal oxidation state=+2.66), preferably a charge of between +2 (average formal oxidation state=+2.33) and +3 (average formal oxidation state=+2.5), more preferably a charge of +2 (average formal oxidation state=+2.33).

Chalcogenolate ligands of the form R-E, (where E is O, S, Se and Te and R is alkyl or aryl), in some cases constitute the bridging ligands in multi-center metal clusters and may form stable complexes with tantalum. The R-groups may serve as attachment points for organic ionic or nonionic polar solubilizing groups, if desired.

Tantalum clusters and nanoparticles have been prepared via a number of different synthetic routes. Methods that are contemplated herein include solid state methods, plasma based synthesis routes, laser ablation, magnetron sputtering, and self propagating high temperature synthesis (SHS) based routes.

A typical solid state synthetic route begins with the reaction of tantalum(V) chloride with tantalum metal powder in the presence of sodium chloride at ˜700° C. in a sealed quartz tube followed by digestion in water, and an acidic workup. Tantalum clusters may also be prepared by aluminum reduction of tantalum(V) chloride in sodium aluminum chloride. Subsequent steps such as ion exchange, ligand exchange, and purifications are accomplished using solution-based inorganic and organometallic methods. Nearly all of the existing tantalum cluster syntheses have focused on the chlorides, and there has been very little work with the bromides or iodides. However, the existing synthetic procedures can be adapted to prepare and isolate bromides and iodides. The stoichiometry and cluster charge are dictated by the average formal oxidation state of the tantalum atoms. Many of the syntheses start with pentavalent tantalum, and following reduction, yield tantalum clusters with average formal oxidation states between di- and trivalency.

The controlled air-oxidation of the standard 16 electron-center clusters (formal oxidation state +2.33) allows oxidation to 14 electron-center clusters (formal oxidation state: 2.66) using methanolic sodium hydroxide. These materials may then be reduced to 15 electron-center clusters (formal oxidation state: 2.5), and eventually back to 16 electron-center clusters with tin(II) chloride. Iron(III) chloride can be used to oxidize the 16 electron-center cluster only as far as the 15 electron-center cluster. The 16 electron-center cluster appears to be the most stable, but the oxidized forms were not evaluated in biological buffers.

In various embodiments, tantalum cluster syntheses were based on high temperature redox reactions. Preliminary clusters may be prepared by the reduction of tantalum(V) chloride with aluminum or a flux-based route with tantalum metal. The clusters prepared via the flux-based reduction are more rapidly extracted, and the overall synthesis is safer and more manageable. The literature approach was significantly off-stoichiometry, so the synthesis was adjusted to enhance efficiency and yield. The syntheses described herein were conducted by reducing tantalum(V) chloride with tantalum metal, at a Ta:TaCl₅ mole ratio of 1.1 to 4.6, preferably 1.1 to 1.5. The material resulting from the reduction of tantalum(V) chloride with tantalum metal is a double salt [Na₄(Ta₆Cl₁₂)Cl₆], including a cluster that dissolves rapidly in water releasing the cluster cation (Ta₆Cl₁₂)⁺².

In an example tantalum cluster synthesis using the flux-based route, tantalum(V) chloride, 3.009 g, tantalum powder, 1.737 g, and sodium chloride, 0.701 g, were ground together with a pestle in a mortar in an inert atmosphere glove box. The mixture was loaded into a 30 cm long, 15 mm ID quartz tube sealed on one end, and fitted to a Whitey ball valve through a Cajun Ultra-Torr fitting. The closed assembly was removed from the glove box, evacuated to <40 mTorr, and sealed with a hydrogen-oxygen torch. The final tube length was 23.5 cm to give a 3.0% fill volume. Enclosed in an open-ended stainless steel tube for protection, the sealed quartz tube was heated at a rate of 2 C.°/min to 700° C. in a box furnace. After a 12-hour dwell, heating was ceased, and the contents were allowed to slowly cool to room temperature over one day. As seen in Table I below, heating the sealed quartz tube at a rate of 2 C.°/min to 850° C. is not recommended, as it produces clusters which have a severely reduced solubility.

The reaction product recovered from the above reaction contains the desired tantalum cluster, sodium chloride, tantalum(V) chloride, and tantalum metal. This mixture is then added to sufficient water to dissolve the tantalum clusters and the sodium chloride. The tantalum(V) chloride is hydrolyzed into insoluble tantalum oxides. The aqueous mixture is filtered to remove tantalum metal and tantalum oxides. Concentrated hydrochloric acid is added to the filtrate to precipitate the crude tantalum clusters. The crude clusters are isolated by filtration or centrifugation. The isolated solid tantalum clusters contain excess chloride anions from sodium chloride and hydrochloric acid contaminants. The solubility of the tantalum clusters is significantly limited in water and other protic solvents by this additional chloride. Specifically, the crude tantalum clusters have a solubility in water of less than 100 mM. The isolated solid tantalum clusters may be purified by washing the clusters with concentrated hydrochloric acid to remove residual sodium chloride or by dissolving the clusters in water followed by precipitating the clusters while leaving sodium chloride in solution. The washed or precipitated clusters are then washed with diethyl ether to remove residual hydrochloric acid. This process of washing or precipitating tantalum clusters at least once, preferably several times, with concentrated hydrochloric acid, and then washing several more times with diethyl ether provides purified tantalum clusters having a solubility in water of at least 120 mM, preferably 150 mM, more preferably 300 mM. The purified tantalum clusters also have a high solubility in ethanol and ethylene glycol. Specifically, the purified tantalum clusters have a solubility in ethylene glycol of at least 150 mM, preferably 300 mM, more preferably 500 mM. The process is of purification may be iterative. In various embodiments, the process involves at least one aqueous hydrochloric acid washing, preferably a plurality of aqueous hydrochloric acid washings, to remove excess sodium from the tantalum clusters as sodium chloride since the sodium chloride is not soluble in ether, but is slightly soluble in concentrated hydrochloric acid (35% HCl in water). The end point for removal of sodium chloride may be detected with a sodium ion-selective electrode to determine when substantially all sodium has been removed. Once the sodium chloride is removed, the hydrochloric acid removal is initiated by repeated washes with diethyl ether. The completion of hydrochloric acid removal may be detected by monitoring the pH of the diethyl ether washings with pH paper or an electrochemical pH sensor to determine when the diethyl ether washings are substantially neutral. Two additional washings were conducted once the pH paper or pH sensor no longer showed an excess of hydronium ions in the evaporated and moistened diethyl ether fractions.

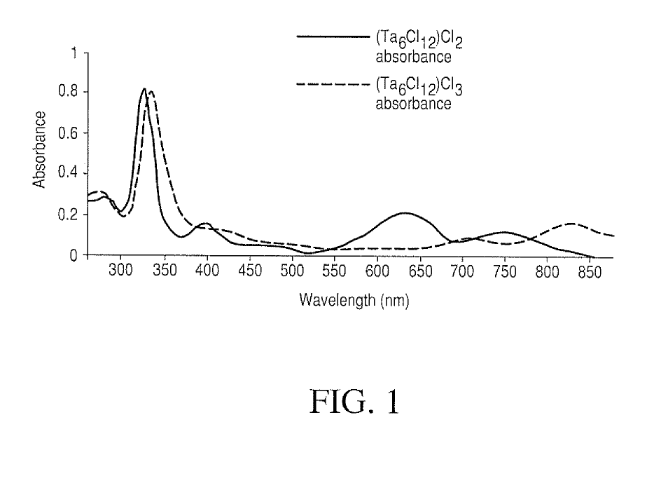

The prototypical 16 electron-center stabilized cluster cation is (Ta₆Cl₁₂)⁺² with a formal tantalum oxidation state of 2.33, and it has been demonstrated that this cluster is easily oxidized to the 15 electron-center cluster (Ta₆Cl₁₂)⁺³ with a formal tantalum oxidation state of 2.5 through the use of the mild oxidizer, iron(III) chloride. The optical absorbance of this cluster changes dramatically, as shown in FIG. 1, where the optical absorbance spectrum of an aqueous solution of (Ta₆Cl₁₂)Cl₂ is shown with a solid line and the optical absorbance spectrum of an aqueous solution of (Ta₆Cl₁₂)Cl₃ is shown with a dashed line.

Targeting a tantalum cluster on par with iodinated imaging agents, a simple calculation for a hexanuclear tantalum cluster revealed that cluster concentrations of greater than 70 mM, preferably greater than 120 mM, more preferably greater than 150 mM, most preferably greater than 300 mM, were desired. Optical absorbencies at ˜330 and ˜400 nm were used to track cluster concentration in solution. An approximate molar extinction coefficient was determined by dissolving an isolated and purified cluster sample in water and measuring its absorption. The molar extinction coefficient, ε at 400 nm is 2263 M⁻¹cm⁻¹. Early in the study, the highest sample concentration was 30 mM. Elemental analysis was performed by Galbraith Laboratories by inductively coupled plasma, optical emission spectroscopy (ICP-OES) and gave a molar extinction coefficient of 4427 M⁻¹cm⁻¹ in reasonable agreement with the approximate gravimetric analysis.

Substitutional doping of divalent zinc on trivalent tantalum sites provides for a reduction in the overall cluster charge. A reduction in the overall cluster charge is also attainable by adjusting the redox chemistry of the starting mixture. Target materials include (Ta₆Cl₁₂)Cl₂, (Ta₆Br₁₂)Cl₂, (TaI₁₂)Cl₂, (Ta₆Br₁₂)Br₂, (Ta₆I₁₂)I₂, (Ta₆Cl₁₁O)Cl₂, (Ta₆Cl₁₁S)CI₂, and (Ta₅ZnCl₁₂)Cl₂. Observing differences in the solubility of samples with different targeted tantalum to tantalum(V) chloride ratios, a panel of compounds was prepared using reactants in a Ta:TaCl5 mole ratio ranging from 1.1 to 4.6. These compositions were targeted to elicit materials with enhanced solubility or molecular handles that could be leveraged to further enhance the solubility. Material properties including color, solubility, and absorption spectra varied from material to material as tabulated below in Table 1 for solubility. In the table below, EtOH stands for ethyl alcohol, EG stands for ethylene glycol, PS80 stands for Polysorbate 80, DMSO stands for dimethyl sulfoxide, and EDTA stands for ethylenediaminetetraacetic acid.

TABLE 1
Cluster concentration (mM) from saturated
solutions in a variety of solvents.

| | Ta: | | | | 10% | | 5% |
|---|---|---|---|---|---|---|---|
| Sample | TaCl5 | H2O | EtOH | EG | PS80 | DMSO | EDTA |
| Ta Red, | 1.2 | 44 | 99 | 87 | 52 | 3 | 12 |
| S-doped | | | | | | | |
| Ta Red, | 1.1 | 0 | 53 | 24 | 48 | 5 | 8 |
| 0-doped | | | | | | | |
| Ta Red, | 1.1 | 34 | 81 | 66 | 54 | 2 | 4 |
| S-doped | | | | | | | |
| Ta Red @ | 4.6 | 4 | | | | | |
| 850 C. | | | | | | | |
| Ta Red | 4.6 | 18 | 46 | 44 | 35 | | |
| Ta Red | 4.0 | 34 | 29 | 49 | 50 | | |
| Ta Red | 2.3 | 42 | 42 | 69 | 71 | | |
| Ta Red | 1.5 | 70 | 66 | 80 | 95 | 1 | 20 |
| Ta Red | 1.2 | 59 | 83 | 95 | 86 | 2 | 12 |
| Ultra | 1.2 | 355 | | 592 | | | |
| purified | | | | | | | |
| Ta Red | | | | | | | |
| Ta Red, Zn | 2.0 | 42 | 22 | 13 | 49 | 1 | 11 |
| Ta Red, Zn* | 1.1 | 1 | 0 | | | | |

The UV-Vis shows that the 2+ and 3+ clusters are distinct, with likely 2.33 and 2.5 formal tantalum oxidation states. All clusters in the above table were obtained by reduction of tantalum(V) chloride by a flux-based route with tantalum metal (Ta Red=tantalum reduced).

As seen in Table 1 above, the solubilities of these complexes were tested in four biocompatible solvents, including water, a 10% aqueous solution of Polysorbate 80, a 5% aqueous solution of ethylenediamine tetraacetic acid (EDTA), and dimethyl sulfoxide (DMSO); as well as the polar solvents ethanol and ethylene glycol. It was found that none of the tantalum clusters showed significant solubility in DMSO, and all showed a solubility of less than 25 mM in a 5% aqueous solution of EDTA. The solubility of (Ta₆Cl₁₂)Cl₂ in an aqueous solution of saturated polyvinyl alcohol) (PVA) was investigated by stirring purified (Ta₆Cl₁₂)Cl₂ with a solution of saturated PVA (MW-50k-85k, hydrolyzed). A green solution resulted, but over time, most of the color was lost as a deep green precipitate formed. It appeared that PVA complexes of tantalum clusters were poorly soluble in water.

However, Ta Red, Ta:TaCl₅=2.3 salts showed a solubility of 70 mM or greater in a 10% aqueous solution of Polysorbate 80. Also Ta Red, Ta:TaCl₅=1.5 showed a solubility of 70 mM or greater in water, ethylene glycol, and a 10% aqueous solution of Polysorbate 80. Further, Ta Red, Ta:TaCl₅=1.2 showed a solubility of 70 mM or greater in ethanol, ethylene glycol, and a 10% aqueous solution of Polysorbate 80. Zinc- or oxygen-doping of tantalum clusters was not found to enhance solubility, but sulfur-doping produced clusters having a solubility of greater than 70 mM in ethanol or ethylene glycol. Thus, Ta Red, S-doped; Ta Red, O-doped; and Ta:TaCl₅=2.3, 1.5, and 1.2 were chosen for further study.

It has been found that isolated solid tantalum clusters having a high solubility of greater than 120 mM, preferably greater than 150 mM, and more preferably greater than 300 mM, in water may be obtained by washing the tantalum clusters with concentrated hydrochloric acid at least once, preferably a plurality of times, to remove all traces of sodium chloride. The washed clusters are then washed with diethyl ether a plurality of times to remove all traces of hydrochloric acid. This process was necessary to obtain purified tantalum clusters having a solubility in water of at least 120 mM, preferably 150 mM, and more preferably 300 mM. The resulting clusters also have a solubility in ethylene glycol of at least 150 mM, preferably 300 mM, and more preferably 500 mM. As seen in Table 2, purification by repeated sequential washing of a tantalum cluster of formula (Ta₆Cl₁₂)Cl₂ (Ta Red, Ta:TaCl₅=1.2) with concentrated hydrochloric acid to remove residual sodium chloride, and with diethyl ether to remove residual hydrochloric acid, produced a tantalum cluster of formula (Ta₆Cl₁₂)Cl₂ (Ultra Purified Ta Red, Ta:TaCl₅=1.2) having a solubility in water of 355 mM and a solubility in ethylene glycol of 592 mM.

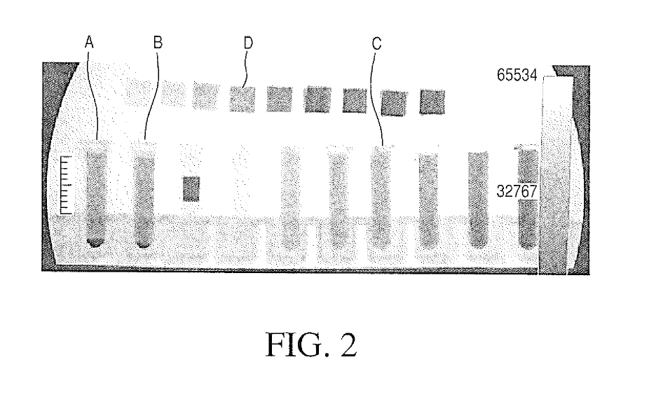

An ultrapurified tantalum cluster of formula (Ta₆Cl₁₂)Cl₂ (Ultra Purified Ta Ta:TaCl₅=1.2) was dissolved in water at a concentration of 355 mM, and in ethylene glycol at a concentration of 592 mM. The resulting water solutions were subjected to radiography to gauge their X-ray absorption characteristics. The samples were loaded into 2 mL polypropylene screw cap tubes with a 1 cm inner diameter. These samples were imaged alongside four calibration standards: 1) aluminum step wedge, 2) polycarbonate step wedge, 3) tantalum foils from 0.0127 to 0.5 mm thick, and 4) a water dilution series containing from 0 to 100% by volume of GE Healthcare's iodinated contrast agent, Visipaque, that contains 320 mg I/mL, in the same 2 mL tubes. Images were collected on a GE Healthcare OEC 9900 Elite digital X-ray Imaging System with a Tri-mode Image Intensifier at 50 KeV with filament currents varied from 0.4 to 9.0 mA. FIG. 2 below is a characteristic image of candidate aqueous samples (first two samples from the left) from the left, marked A and B) alongside calibration samples. The most concentrated aqueous samples were unstable as evident in the X-ray opaque precipitates in the samples A and B. However, the remaining soluble cluster absorption is still around that of the 37.5% Visipaque calibration solution (marked C), and a 0.0889 mm thick tantalum foil stack (fourth square from the left, marked D).

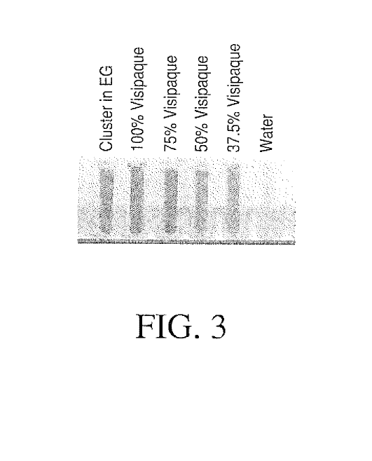

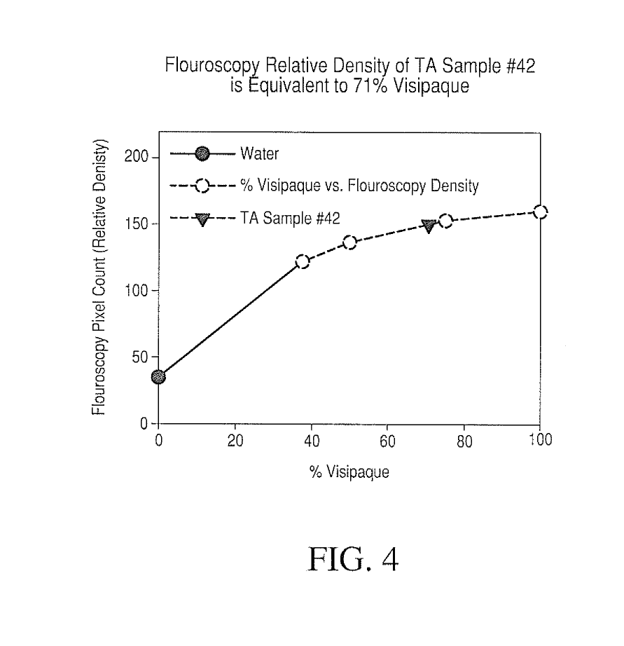

FIG. 3 compares the most concentrated tantalum cluster solution prepared with ethylene glycol side by side with a water standard and a series of Visipaque standard solutions at concentrations ranging from 37.5% to 100%. FIG. 3 is imaged through 8 cm of water serving as a low X-ray energy filter and simulating an arm or leg phantom to produce clinically equivalent images. The absorbencies of water and the Visipaque dilution series yield a calibration curve, shown in FIG. 4, that puts the tantalum cluster solution in ethylene glycol at the opacity of a 71% Visipaque solution. Further contrast enhancement can be expected under X-ray imaging optimized for tantalum as higher energy X-rays can be effectively utilized.

Various embodiments of the current invention relate to a method of obtaining an enhanced X-ray image of a living subject by administering an X-ray contrast agent comprising a saturated solution of multinuclear tantalum clusters having bridging ligands to the living subject. The X-ray contrast agent to may be administered to the living subject by intravenous or intra-arterial injection, or by other known methods of administering a contrast agent. Procedures for administration of a solution of a metal-based X-ray contrast agent by injection into the circulatory system to enhance X-ray contrast in a subject are known in the art.

Although the various exemplary embodiments have been described in detail with particular reference to certain exemplary aspects thereof, it should be understood that the invention is capable of other embodiments and its details are capable of modifications in various respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A solution comprising a defined concentration of tantalum clusters having the formula (Ta₆₋ₓMₓX₁₂)L_{y}A_{z}, where:
M is selected from the group consisting of zinc, niobium, and tungsten;
X is selected from the group consisting of chlorine, bromine, iodine, oxygen, sulfur, selenium, and tellurium;
L is an inorganic ligand or an organic ligand which is charged or uncharged;
A is an inorganic counterion;
x is between 0 and 6;
y is between 0 and 6; and
z is selected so as to maintain electrical neutrality of the clusters;
said solution comprising a solvent selected from the group consisting of water, an aqueous solution of Polysorbate 80, ethanol, ethylene glycol, and propylene glycol;
wherein said defined concentration is greater than 120 mM.

2. The solution of claim 1, where x is zero.

3. The solution of claim 2, wherein A is Cl, Br, or I.

4. The solution of claim 2, wherein each X is CI, Br, or I.

5. The solution of claim 2, wherein each X is Cl.

6. The solution of claim 2, wherein the tantalum clusters have the formula (Ta₆Cl₁₂)Cl₂.

7. The solution of claim 2, wherein said tantalum clusters have the formula (Ta₆Cl₁₂)A_{z}, where A is Cl and z is between 2 and 3.

8. The solution of claim 7, wherein the solvent is water or an aqueous solution of Polysorbate 80.

9. The solution of claim 8, wherein the defined concentration is greater than 150 mM.

10. The solution of claim 8, wherein the defined concentration is greater than 300 mM.

11. The solution of claim 7, wherein the solvent is ethylene glycol or propylene glycol.

12. The solution of claim 11, wherein the defined concentration is greater than 150 mM.

13. The solution of claim 11, wherein the defined concentration is greater than 300 mM.

14. The solution of claim 11, wherein the defined concentration is greater than 500 mM.

15. The solution of claim 7, wherein the solvent is ethanol.

16. A process for purifying tantalum clusters having the formula (Ta₆₋ₓMₓX₁₂)L_{y}A_{z}, wherein:
M is selected from the group consisting of zinc, niobium, and tungsten;
X is selected from the group consisting of chlorine, bromine, iodine, oxygen, sulfur, selenium, and tellurium;
L is an inorganic ligand or an organic ligand which is charged or uncharged;
A is an inorganic counterion;
x is between 0 and 6;
y is between 0 and 6; and
z is between 2 and 3;
said process comprising:
a) preparing crude tantalum clusters;
b) washing the crude tantalum clusters at least once with aqueous hydrochloric acid to remove residual sodium chloride;
c) washing the crude tantalum clusters with diethyl ether to remove residual hydrochloric acid and water; and
d) repeating step (c) a plurality of times until substantially all hydrochloric acid is removed from said crude tantalum clusters; and
e) recovering purified tantalum clusters having a solubility in water of greater than 120 mM.

17. The method of claim 16, wherein said crude tantalum cluster are obtained by dissolving a mixture of tantalum clusters, sodium chloride, tantalum metal, and tantalum oxides in water to form a solution; filtering the solution to remove insoluble solids; precipitating the tantalum clusters with concentrated hydrochloric acid; and recovering the precipitated solids.

18. Purified tantalum clusters having a solubility in water of greater than 100 mM, said purified tantalum clusters having the formula (Ta₆₋ₓMₓX₁₂)L_{y}A_{z}, wherein:
M is selected from the group consisting of zinc, niobium, and tungsten;
X is selected from the group consisting of chlorine, bromine, iodine, oxygen, sulfur, selenium, and tellurium;
L is an inorganic ligand or an organic ligand which is charged or uncharged;
A is an inorganic counterion;
x is between 0 and 6;
y is between 0 and 6; and
z is between 2 and 3;
wherein said purified tantalum clusters are obtained by a process comprising:
a) preparing crude tantalum clusters;
b) washing the crude tantalum clusters at least once with aqueous hydrochloric acid to remove residual sodium chloride;
c) washing the crude tantalum clusters with diethyl ether to remove residual hydrochloric acid and water; and
d) repeating step (c) a plurality of times until substantially all hydrochloric acid is removed from said crude tantalum clusters; and
e) recovering purified tantalum clusters having a solubility in water of greater than 120 mM.

19. Tantalum clusters having a solubility in an aqueous solution of 10% Polysorbate 80 of greater than 70 mM, said tantalum clusters having the formula (Ta₆₋ₓMₓCl₁₂)L_{y}A_{z}, wherein:
M is selected from the group consisting of zinc, niobium, and tungsten;
X is selected from the group consisting of chlorine, bromine, iodine, oxygen, sulfur, selenium, and tellurium;
L is an inorganic ligand or an organic ligand which is charged or uncharged;
A is an inorganic counterion;
x is between 0 and 6;
y is between 0 and 6; and
z is between 2 and 3;

20. A saturated solution, comprising:
a solvent, said solvent being an aqueous solution of 10% Polysorbate 80; and
a solute, said solute comprising tantalum clusters according to claim 19.

21. An X-ray contrast agent, comprising a concentrated solution of multinulear tantalum clusters having bridging ligands;
said concentrated solution having a concentration of greater than 70 mM.

22. An X-ray contrast agent of claim 21, wherein said bridging ligands are halide ions.

23. An X-ray contrast agent of claim 21, wherein said concentrated solution has a concentration of greater than 120 mM.

24. An X-ray contrast agent of claim 21, wherein said concentrated solution has a concentration of greater than 150 mM.

25. An X-ray contrast agent of claim 21, wherein said concentrated solution has a concentration of greater than 300 mM.

26. A method of obtaining an enhanced X-ray image of a subject, comprising administering the X-ray contrast agent of claim 21 to said subject.

27. A method of obtaining an enhanced X-ray image of a living subject, comprising administering the X-ray contrast agent of claim 21 to said living subject by intra-arterial injection.
